(19) United States
(12) Patent Application Publication
(10) Pub. No.: US 2020/0069391 A1
(43) Pub. Date: Mar. 5, 2020

(54) MULTI-BALLOON POUCH FORMING DEVICE

(71) Applicant: BOSTON SCIENTIFIC SCIMED, INC., MAPLE GROVE, MN (US)

(72) Inventors: MATTHEW MCEVADDY, GALWAY (IE); AIDEN FLANAGAN, CO. GALWAY (IE); MARTIN LAWRENCE FAWDRY, GALWAY (IE); CLARA GRACE JULIA HYNES, CO. DUBLIN (IE)

(73) Assignee: BOSTON SCIENTIFIC SCIMED, INC., MAPLE GROVE, MN (US)

(21) Appl. No.: 16/552,741

(22) Filed: Aug. 27, 2019

Related U.S. Application Data
(60) Provisional application No. 62/724,362, filed on Aug. 29, 2018.

(51) Int. Cl. A61B 90/00; A61M 25/10; A61M 29/02

(52) U.S. Cl. CPC A61B 90/02; A61M 25/1002; A61M 2210/1021; A61M 29/02; A61M 25/1011

(57) ABSTRACT

An inflation device may include a plurality of independently inflatable balloons attached to one another. The balloons may be configured to move between a deflated, compressed state and an inflated, expanded state. The inflation device further includes a plurality of inflation tubes connected to the plurality of inflatable balloons, wherein each inflation tube is attached to a single one of the plurality of inflatable balloons.

### CROSS-REFERENCE TO RELATED APPLICATIONS

This application claims the benefit of priority under 35 U.S.C. § 119 to U.S. Provisional Application Ser. No. 62/724,362, filed Aug. 29, 2018, the entirety of which is incorporated herein by reference.

The work leading to this invention has received funding from the European Union's Diabetes Reversing Implants with enhanced Viability and long-term Efficacy Project (“DRIVE”) (call identifier H2020-NMP10-2014) under grant agreement no. 645991—DRIVE.

### TECHNICAL FIELD

The present disclosure pertains to medical devices, and methods for manufacturing medical devices. More particularly, the present disclosure pertains to medical devices for forming a pouch between layers of tissue.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example medical device includes an inflation device comprising a plurality of independently inflatable balloons attached to one another, the balloons configured to move between a deflated, compressed state and an inflated, expanded state, and a plurality of inflation tubes connected to the plurality of inflatable balloons, wherein each inflation tube is attached to a single one of the plurality of inflatable balloons.

Alternatively, or additionally to the embodiment above, the inflation device further comprises a plurality of valves configured to control inflation and deflation of each balloon independently, wherein the valves are disposed either within each balloon or within each inflation tube.

Alternatively, or additionally to the embodiment above, the valves are disposed within each balloon.

Alternatively, or additionally to the embodiment above, each balloon defines a to single internal inflation chamber.

Alternatively, or additionally to the embodiment above, the plurality of inflatable balloons includes a center balloon and at least one laterally outward balloon attached to either side of the center balloon, wherein the internal inflation chamber of the center balloon has a generally circular cross-section and the internal inflation chambers of the is laterally outward balloons have an elliptical cross-section.

Alternatively, or additionally to the embodiment above, the elliptical cross-section has a major dimension greater than a diameter of the center balloon internal chamber, and wherein the major dimension increases in each successively further laterally outward balloon.

Alternatively, or additionally to the embodiment above, the plurality of inflatable balloons each have a distal end, a proximal end, and a side surface, wherein the inflation tubes are attached to the proximal ends and adjacent balloons are attached to one another along their side surfaces.

Alternatively, or additionally to the embodiment above, the plurality of inflatable balloons are attached to one another in a plane, with each balloon attached to no more than two adjacent balloons.

Alternatively, or additionally to the embodiment above, the plurality of inflatable balloons are arranged with a center balloon distal end extending further distally than distal ends of adjacent balloons.

Alternatively, or additionally to the embodiment above, the plurality of inflatable balloons are compliant.

Alternatively, or additionally to the embodiment above, the inflation device further comprises at least one guidewire lumen extending through one of the plurality of inflatable balloons or extending through a region of attachment between adjacent balloons.

Alternatively, or additionally to the embodiment above, the inflation device further comprises an outer sleeve disposed around the plurality of inflatable balloons.

Alternatively, or additionally to the embodiment above, the inflation device further comprises a connector element configured to attach the inflation device to a body lumen or organ, wherein the connector element connects distal ends of the inflatable balloons to proximal ends of the inflatable balloons or the connector element connects a side surface of a leftmost balloon to a side surface of a rightmost balloon.

Alternatively, or additionally to the embodiment above, the plurality of inflatable balloons defines at least a 2×2 grid of balloons.

Another example inflation device comprises a plurality of laterally adjacent independently inflatable balloons configured to be expanded from a collapsed delivery configuration to an expanded configuration having a generally flattened profile, the inflatable balloons including a series of longitudinally-oriented elongate balloons attached to one another along side surfaces thereof, the balloons including a center balloon and at least two side balloons attached to opposite sides of the center balloon, a plurality of inflation tubes, each tube connected to one of the plurality of balloons, and at least one guidewire lumen extending through one of the plurality of inflatable balloons or extending through a region of attachment between adjacent balloons.

Alternatively, or additionally to the embodiment above, the inflation device further comprises a plurality of valves configured to control inflation and deflation of each balloon independently, wherein the valves are disposed either within each balloon or within each inflation tube.

Alternatively, or additionally to the embodiment above, the inflation tubes are attached to proximal ends of each balloon.

Alternatively, or additionally to the embodiment above, the plurality of inflatable balloons are arranged with a distal end of the center balloon extending further distally than distal ends of the at least two side balloons.

Alternatively, or additionally to the embodiment above, further comprising an outer sleeve disposed around the plurality of inflatable balloons.

Another example inflation device comprises a catheter with a lumen extending therethrough, the catheter having a hub disposed at a proximal end thereof, the hub including a seal, a plurality of independently inflatable balloons permanently attached to one another, the balloons configured to move between a deflated, compressed state when inside the catheter, and an inflated, expanded state when released from the catheter, a plurality of inflation tubes connected to the plurality of inflatable balloons, wherein each inflation tube is attached to a single one of the plurality of inflatable balloons, and an outer sleeve disposed around the plurality of inflatable balloons when the balloons are disposed within the hub, wherein the seal is configured to retain the outer sleeve in the hub as the balloons are moved through the seal.

An example method of forming a pouch within an abdominal wall comprises inserting an inflation device between a first layer of tissue of the abdominal wall and a second layer of tissue of the abdominal wall immediately adjacent the first layer of tissue, the inflation device comprising a plurality of independently inflatable balloons attached to one another, the balloons configured to move between a deflated, compressed state and an inflated, expanded state, and a plurality of inflation tubes connected to the plurality of inflatable balloons, wherein each inflation tube is attached to a single one of the plurality of inflatable balloons. The method further includes positioning the inflation device at a desired location of the pouch within the abdominal wall, selectively and individually inflating the inflatable balloons within the abdominal wall, thereby separating the first layer of tissue from the second layer of tissue to form the pouch within the abdominal wall, deflating the inflatable balloons within the abdominal wall, and withdrawing the inflation device from the abdominal wall.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a partial anterior cut-away view of an abdomen of a human body;

FIG. 2 is a partial cross-sectional view of an anterior wall of an abdomen of a human body, taken along line 2-2 of FIG. 1;

FIG. 3 illustrates an example inflation device in a collapsed delivery configuration;

FIG. 4 illustrates an example inflation device in an inflated, expanded configuration;

FIG. 5 is a cross-sectional view of an example inflation device;

FIG. 6 is a cross-sectional view of another example inflation device;

FIG. 7 is a side cross-sectional view of an example inflation device within a catheter hub;

FIGS. 8A-8C are cross-sectional views of an example inflation device in various stages of inflation;

FIGS. 9A-9C are cross-sectional views of another example inflation device in various stages of inflation;

FIG. 10 is a cross-sectional view of an example inflation device disposed over a body lumen;

FIGS. 11A and 11B illustrate example inflation devices attached to a heart; and

FIG. 12 illustrates an example inflation device.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” is may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g. 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Some diseases may negatively affect the quality of life of various people all over the world. Some diseases may be chronic, lifelong illnesses requiring constant treatment and/or intervention. For example, diabetes results from the pancreas not producing enough insulin or the cells of the body not properly responding to the insulin produced. Some diabetes patients require insulin injections or other medications to manage their condition and/or to reduce complications. Promising new or alternative therapies continue to be developed. Disclosed herein are minimally-invasive devices for forming a pouch in an abdominal wall to allow insertion and/or implantation of a diabetes-reversing implant or similar device in an area that is relatively easily accessed surgically and is highly vascularized. The disclosed devices may also be used to form pouches or tunnels within the body to aid in the placement of leads, electrodes, or other devices between skin layers, between muscle layers, and beneath the sternum. In other embodiments, the disclosed devices may be used to access the pericardial or pleural cavities, for opening adhesions, and for providing surgical access in obese patients. In addition to aiding in the implantation of diabetes-reversing implants, the disclosed devices may be used as an epicardial heart pump, an extra- or intra-aortic pump, and as a peristalsis enhancement device in the gastrointestinal tract.

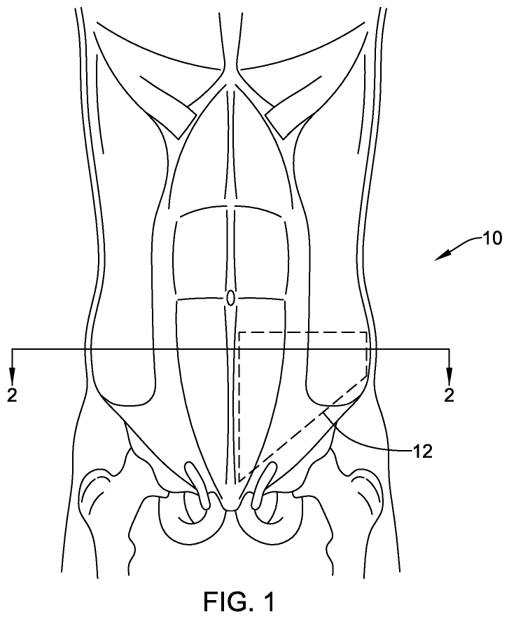

FIG. 1 illustrates a partial cut-away anterior view of the abdominal area 10 of the human body. In some embodiments, a potential shell space 12 in which to form an abdominal pouch may be located on a left side of the body (e.g., laterally to the left of the midsagittal plane). Other locations may also be suitable for various procedures and/or alternatives, and use of the disclosed devices is not limited to the left anterior portion of the body.

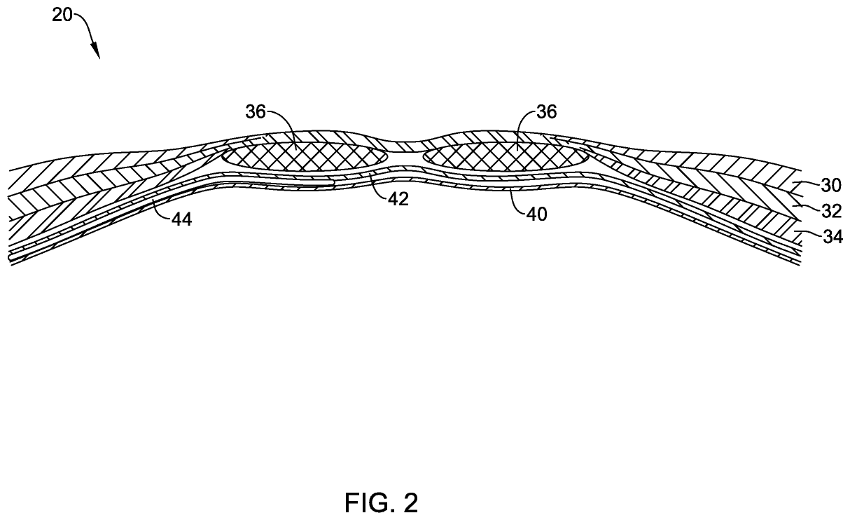

FIG. 2 illustrates a partial cross-section of an anterior abdominal wall 20 of the abdominal area 10 of the human body. Some elements of the abdominal wall 20 may be seen in FIG. 2. The abdominal wall 20 includes three flat muscles situated laterally—the external oblique 30, the internal oblique 32, and the transversus abdominus 34. The abdominal wall 20 includes two vertical muscles situated near the mid-line of the body, the rectus abdominis 36 and the pyramidalis (not visible). In some embodiments, an inflation device may be used to form an abdominal pouch 44 between the tissue layers of the parietal peritoneum 40 and the transversalis fascia 42. This area of tissue layers lies beneath the abdominal muscles and on top of (e.g., anterior of) the intestines in the pubic and/or left groin areas of the abdomen.

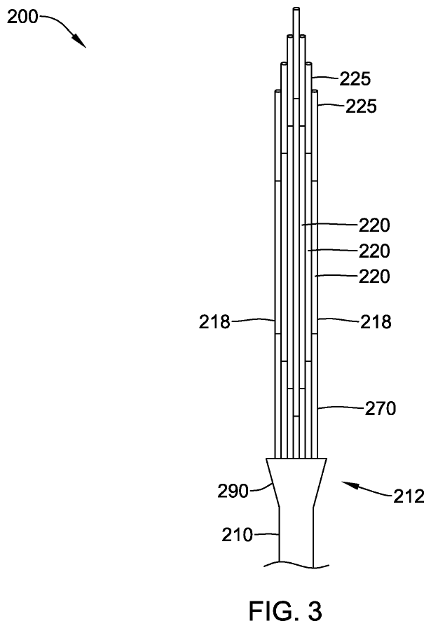

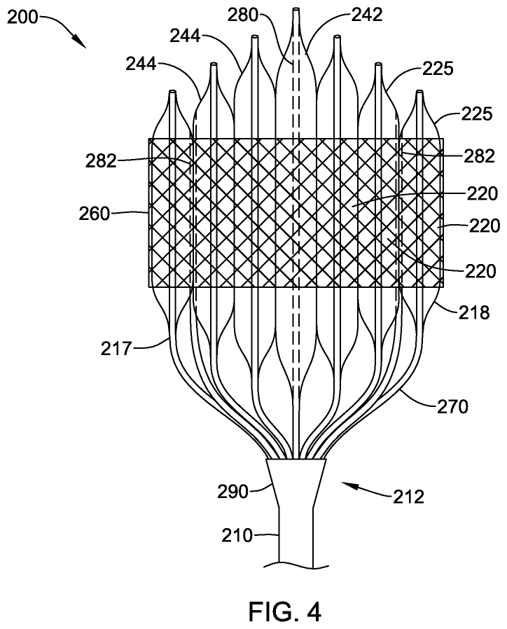

An example inflation device 200 for forming an abdominal pouch may include a plurality of independently inflatable balloons 220 configured to be delivered through a distal end 212 of a catheter 210, as seen in FIGS. 3 and 4. FIG. 3 illustrates the inflation device 200 in a deflated or compressed state and FIG. 4 illustrates the inflation device 200 in an inflated or expanded state.

In some embodiments, the inflation device 200 may include a plurality of separately formed and independently inflatable balloons 220 permanently attached to one another with adhesive, heat bonding, or other suitable connecting elements. In other embodiments, the inflation device 200 may be formed initially as a single large balloon that is then heat sealed to create multiple longitudinal and independently inflatable balloons 220. Each of the balloons 220 may be inflated and deflated separately and independently of the other balloons 220. The balloons 220 may be attached to one another in a plane, with each balloon attached to no more than two adjacent balloons. In this manner, the inflation device 200 is a planar structure with a thickness of only a single balloon 220. The inflatable balloons 220 may be attached to adjacent balloons along their side surfaces, as shown in FIG. 4.

In some embodiments, the distal ends of the inflatable balloons 220 may include a distally tapered portion 225, and proximal ends of the inflatable balloons 220 may include a proximally tapered portion 218. The proximally tapered portion 218 of each inflatable balloon 220 may be coupled to a separate inflation tube 270 that may be coupled to a catheter 210. In some embodiments, the catheter 210 may have a single lumen and all of the inflation tubes 270 may extend through the lumen to a proximal end the catheter 210 and be coupled to an inflation source. In some embodiments, the proximally tapered portion 218 of each inflatable balloon 220 may be integrally formed with the inflation tube is 270. In other embodiments, the proximally tapered portion 218 may be affixed to the inflation tube 270 with adhesive, heat bonding, or other suitable coupling elements.

In some embodiments, the distal end 212 of the catheter 210 may include a conical element 290 secured to the catheter 210. In some embodiments, the conical element 290 may aid in removal of the inflatable balloons 220 from a target site. In some embodiments, the inflatable balloons 220 may be non-compliant polymeric balloons. In some embodiments, the inflatable balloons 220 may be compliant polymeric balloons having some sort of support structure limiting how far the balloons may stretch and/or extend. Other suitable materials are also contemplated. The inflatable balloons 220 may be expandable from a collapsed delivery configuration, as seen in FIG. 3, to an expanded state, as seen in FIG. 4.

In some embodiments, the size of each of the balloons 220 may be varied in order to create varied shapes of and sizes of pouch 44 and ease of tissue layer separation with reduced trauma. In some embodiments, the distal end of the center balloon 242 may extend further distally than distal ends of the adjacent balloons. The distal ends of the remaining balloons 220 may be incrementally more proximal, creating a tapered overall distal end of the inflation device 200, as seen in FIG. 4. In some embodiments, the center balloon 242 may have a larger diameter and/or length than the laterally outward balloons 244 as shown in FIG. 4, creating an eye shaped pouch with less stretching of tissue at the edges. The length and diameter of each balloon 220 may also be varied creating a disk shaped pouch 44. Each balloon 220 may have a set diameter and length, as with non-compliant balloons, or the diameter and/or length of each balloon 220 may be based on the level of inflation, as with compliant balloons. Compliant balloons may allow a greater variety of inflation patterns as each balloon may be inflated to a different level.

The inflation device 200 may include at least one lumen 280 extending through the interior of one or more of the balloons 220. The lumen 280 may serve as a guidewire lumen, allowing the device to be delivered over a guidewire. The lumen 280 may alternatively allow for a camera, light source, or other instrument to be inserted and utilized during the medical procedure. In some embodiments, the lumen 280 may be disposed through the center balloon 242, as shown in FIG. 4. The lumen 280 may extend alongside the inflation tube 270. In other embodiments, the lumen 280 may serve both as an inflation is lumen and as an instrument lumen, with the lumen 280 including a seal allowing an instrument to pass through without deflating the balloon. In other embodiments, one or more offset lumens 282 may be disposed between adjacent balloons 220. The offset lumens 282 may allow for guidewires, cameras, light sources, or other instruments to be inserted and utilized during the medical procedure. In some embodiments, the offset lumens 282 may be separate tubular structures attached between adjacent balloons 220. In other examples, the offset lumens 282 may extend through the region of attachment between adjacent balloons 220. In embodiments in which the balloons 220 are formed by sealing chambers in a single large balloon, the offset lumens 282 may be formed through the material defining the region of attachment between balloons. In addition to or alternatively to the lumen 280 through a balloon, one or more offset lumens 282 may aid in unwrapping and re-wrapping the inflation device 200. FIG. 4 illustrates two offset lumens 282, one on each side of the center balloon 242.

The inflation and/or deflation of each balloon 220 may be adjusted separately at the time of implantation, or may be adjusted as desired post-implantation. In some embodiments, each balloon 220 may be inflated separately by delivering inflation media through the individual inflation tube 270 attached to the balloon 220. An inflator that remains pressurized may keep the balloons 220 inflated as long as needed. When the balloons 220 are to be removed, the pressure may be released, allowing the inflation fluid to leave the balloons 220. In other embodiments, a plurality of sealing valves 217 may be configured to control the inflation and deflation of each balloon separately. The valves 217 may be disposed at the proximally tapered portion 218 of each balloon 220, as seen in FIG. 4, or within each inflation tube 270 (not shown). In embodiments in which the inflation device 200 is to be a permanent implant, the valves 217 may be disposed at the proximal ends of each balloon 220 such that the inflation tubes 270 may be disconnected and removed from the body, leaving the sealed inflated balloons 220 in the body.

In some embodiments, the inflation device 200 may optionally include an outer sleeve 260 loosely disposed about and/or at least partially surrounding the inflatable balloons 220, as shown in FIG. 4. In some embodiments, at least a portion of the outer sleeve 260 may be bunched up and/or folded between portions of the inflation device 200 in the collapsed delivery configuration (e.g., between folds, between rolled up portions, etc.). In some embodiments, the outer sleeve 260 may be formed from an elastic, resilient, and/or compliant material. In some embodiments, the outer sleeve 260 may be formed from an inelastic or non-resilient material. In some embodiments, the outer sleeve 260 may be an expandable material capable of stretching and/or expanding (e.g., a mesh, a net, a stent-like structure, etc.) from a collapsed delivery configuration to an expanded configuration along with the inflatable balloon 220, as seen in FIG. 4 for example. Some suitable but non-limiting materials for the outer sleeve 260, for example polymeric or metallic materials, are described below.

In use, the outer sleeve 260 may serve to reduce and/or minimize rubbing and/or friction between the inflatable balloons 220 and the tissue layers of the parietal peritoneum 40 and the transversalis fascia 42 when inflating the inflatable balloons 220 to form the abdominal pouch 44. After expanding the inflatable balloons 220 (and the outer sleeve 260) to the expanded configuration and forming the abdominal pouch 44, the inflatable balloons 220 may be deflated and the outer sleeve 260 left behind within the abdominal pouch in engagement with the tissue layers of the parietal peritoneum 40 and the transversalis fascia 42 (e.g., the walls of the abdominal pouch 44) to line the abdominal pouch 44 and help prevent re-adhesion between the tissue layers of the parietal peritoneum 40 and the transversalis fascia 42 until a medical implant or device is positioned in the abdominal pouch 44. In some embodiments, a period of time may pass between formation of the abdominal pouch 44 and implantation of the medical implant or device. Depending on how long the period of time is, the outer sleeve 260 may permit the tissue layers of the parietal peritoneum 40 and the transversalis fascia 42 to recover from inflammation caused by dissection with the inflatable balloons 220 and/or to minimize reaction to the eventual implanted medical implant or device.

In some embodiments, the outer sleeve 260 may include a coating and/or substance disposed on and/or eluting from the outer sleeve 260 which may include a vascular endothelial growth factor (VEGF) and/or other substance configured to stimulate the growth of new blood vessels such that a highly vascularized “bed” is created around the outer sleeve 260 and/or the abdominal pouch 44 to receive the implanted medical implant or device. Similarly, the inflatable balloons 220 may be coated with, inject, release, and/or otherwise deposit a substance (e.g., a gel, a solution, etc.) in the abdominal pouch 44 during and/or after inflation of the inflatable balloons 220 to the expanded configuration to reduce and/or minimize tissue damage and/or fibrosis, and/or to maintain patency of the abdominal pouch 44 for the delivery of the medical implant or device at some later point in time. In some embodiments, the substance may include and/or release pro-angiogenic elements to encourage development of the vascularized “bed” prior to implantation of the medical implant or device.

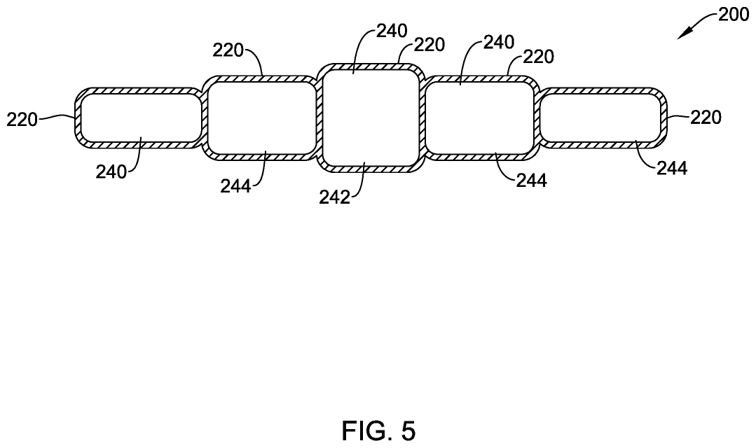

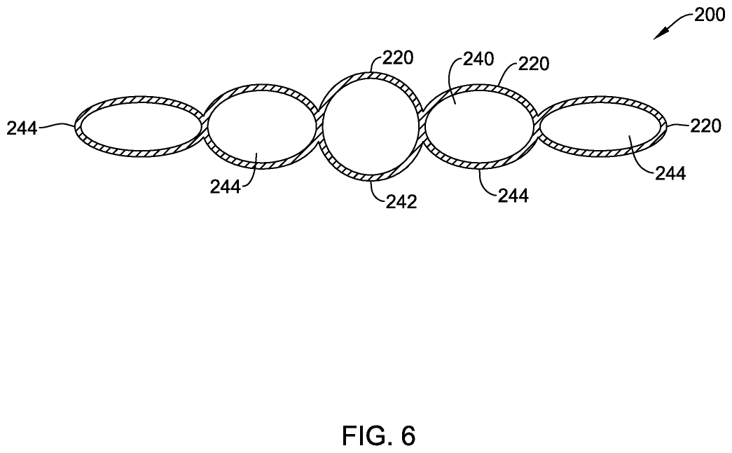

In some embodiments, each balloon 220 in the inflation device 200 may have a different cross-sectional shape and/or size upon inflation. FIGS. 5 and 6 show cross sections through example inflation devices 200. Each of the inflatable balloons 220 may comprise a single internal inflation chamber 240. In other embodiments, each of the inflatable balloons 220 may include a plurality of fluidly connected internal inflation chambers. In some embodiments, the internal inflation chamber 240 of each balloon may have a generally rectangular cross-section, as seen in FIG. 5 for example. As viewed in cross-section, a width of each of the plurality of internal inflation chambers 240 may increase in each successive laterally outward balloon 244 from the center balloon 242, and a height or overall thickness of each of the plurality of internal inflation chambers 240 may decrease in each successive laterally outward balloon 244 from the center balloon 242.

In some embodiments, the center balloon 242 of the plurality of balloons 220 may have a generally circular or round cross-section, and each successive laterally outward balloon 244 may have an elliptical cross-section, as seen in FIG. 6 for example. The elliptical cross-section may have a major dimension greater than a diameter of the center balloon 242, and the major dimension may increase in each successively further laterally outward balloon 244. In other words, as viewed in cross-section, a width of each of the plurality of balloons 220 may increase in each successive laterally outward balloon 244 from the center balloon 242, and a height or overall thickness of each of the plurality of balloons 220 may decrease in each successive laterally outward balloon 244 from the center balloon 242. Generally speaking, a cross-sectional profile of the inflatable balloons 220 may be generally flattened (wider and/or longer than it is tall or thick). In other words, the inflatable balloons 220 may not have a bulbous three-dimensional shape.

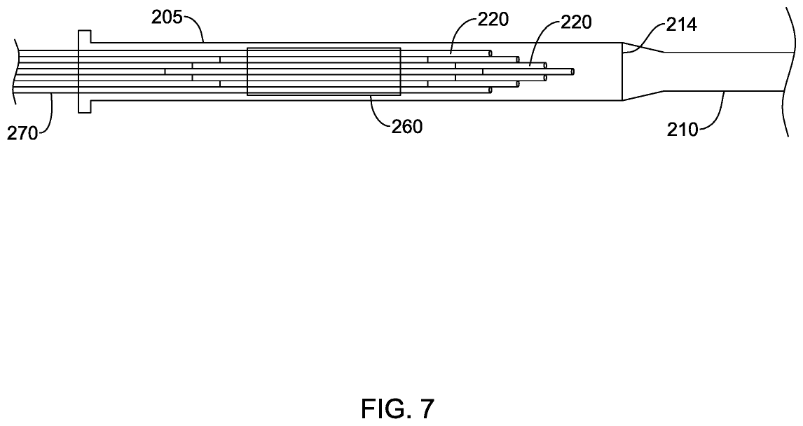

In some embodiments, the outer sleeve 260 may serve as a compression sleeve to keep the inflatable balloons 220 in the compressed state until delivery. As illustrated in is FIG. 7, a hub 205 attached to the proximal end of the catheter 210 may have a hemostasis valve 214 that may engage the outer sleeve 260 and retain the outer sleeve 260 within the hub 205 as the inflatable balloons 220 are moved into and through the catheter 210. In some embodiments, the inflation tubes 270 have sufficient pushability to advance the balloons 220 through the valve and through the catheter 210. In the collapsed delivery configuration, the inflation device 200 may be folded, wrapped, crimped, rolled, coiled, etc. to a smaller overall profile than the expanded configuration to facilitate delivery to and implantation at the target site, such as within the abdominal wall 20. The inflatable balloons 220 may be expandable primarily along their width. In some embodiments, the inflatable balloons 220 may also be expandable along their length.

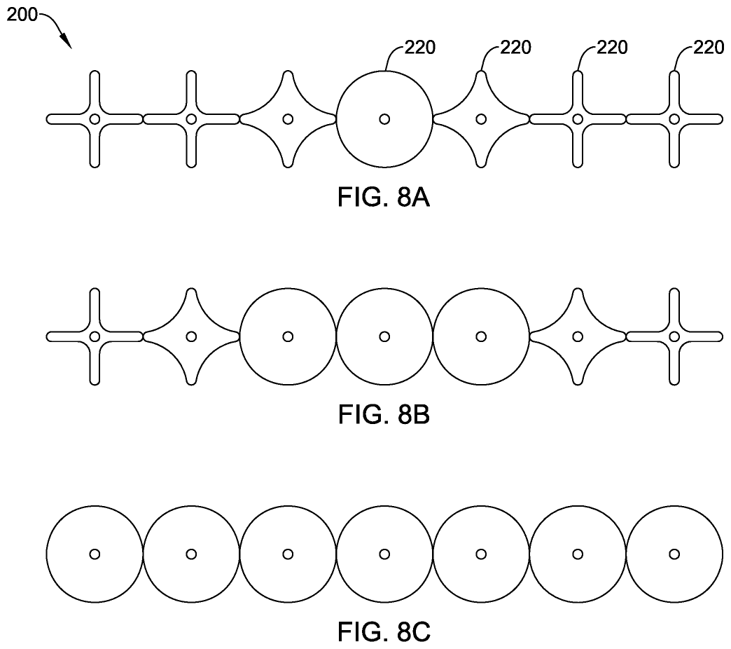

The inflatable balloons 220 may all be individually inflatable in a variety of patterns and degrees of inflation. For example, in an inflation device 200 including seven balloons 220, the center balloon 242 may be inflated first, as shown in FIG. 8A, followed sequentially by the laterally outward balloons 244 adjacent the center balloon 242, as shown in FIG. 8B, and so on until all balloons are inflated, as shown in FIG. 8C. In other embodiments, all of the balloons may be inflated at the same time, or the balloons may be inflated from the outside in towards the center balloon 242, or any other order.

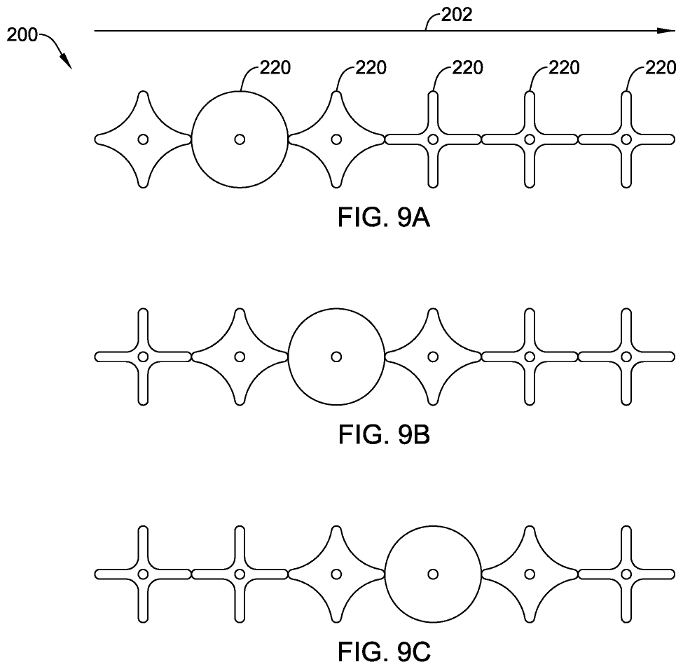

In some embodiments, the inflatable balloons 220 may be inflated independently of one another in a wave type motion and the device may be used as a pump or tissue massage device. For example, in a device with six balloons 220 as shown in FIGS. 9A-9C, the balloons 220 may be inflated in sequence. If the balloons 220 are numbered one to six from left to right in FIGS. 9A-9C, then balloon one could be partially inflated then fully inflated as balloon two is partially inflated, then balloon three may be partially inflated, balloon two fully inflated and balloon one starts to be deflated, as shown in FIG. 9A. FIG. 9B shows the next stage, as balloon four is partially inflated, balloon three is fully inflated, balloon two is partially deflated, and balloon one is fully deflated. FIG. 9C shows the following step, with balloon five partially inflated, balloon four fully inflated, balloon three partially deflated, and balloons two and one completely deflated. In this manner, a wave may be created from left to right, indicated by arrow 202, and the device may be used for situations in which peristalsis type movement is desired. In some embodiments, the peristalsis type movement may be used to supplement the diaphragm is movement and aid in breathing, for example in the event of phrenic nerve damage resulting from spinal cord injury, physical trauma such as a neck injury, or operative trauma and damage occurring unintentionally during heart or abdominal surgery. In such cased, the inflation device 200 may be inserted adjacent the diaphragm.

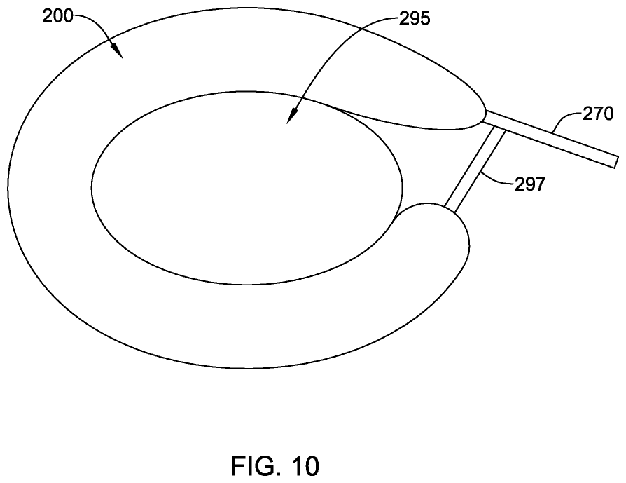

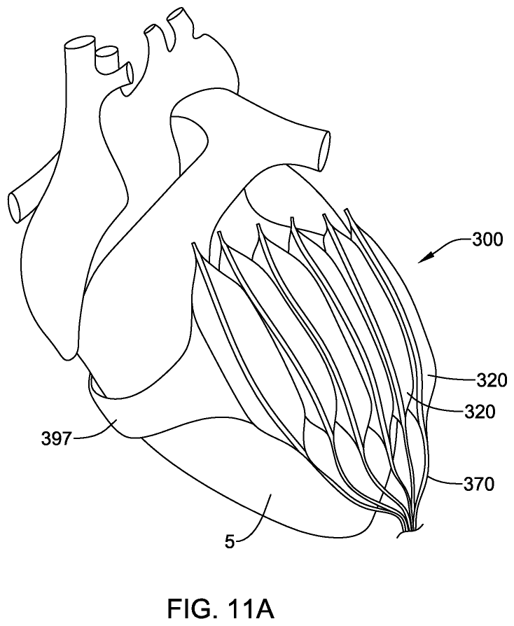

In other embodiments, the peristalsis type movement may be for assisting the movement of fluids through body lumens, such as for aiding blood pumping or aiding movement of food through the digestive tract. FIG. 10 illustrates the inflation device 200 disposed around a body lumen 295. The body lumen 295 may be an artery or a portion of the colon or intestine. A releasable attachment element 297 may be disposed on the distal end of the inflation device 200 and be configured to be attached to the proximal end of the inflation device 200 or to the inflation tubes 270 to keep the device in place around the body lumen 295. The inflation device 200 may be placed around an artery or portion of the digestive tract in a location and orientation such that the sequential inflation/deflation of the balloons 220, as shown in FIGS. 9A-9C, creates a pressure wave that may assist movement of fluid through the body lumen 295.

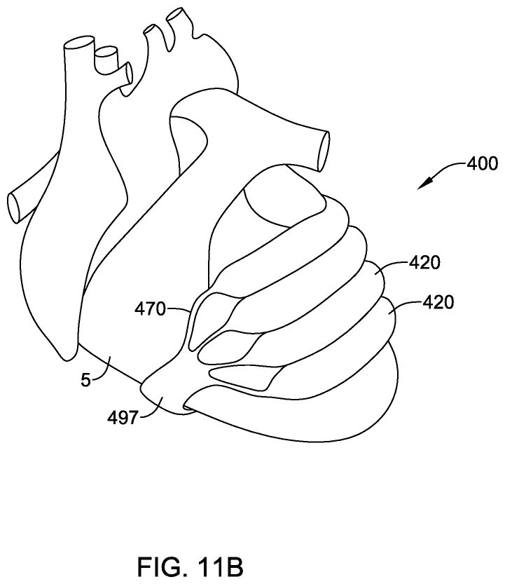

FIGS. 11A and 11B illustrate example inflation devices 300, 400 disposed over a portion of the heart 5 to function as an epicardial pump. As shown in FIG. 11A, a strap 397 may be attached to opposite sides of the inflation device 300 to hold the device in place over the heart 5. The balloons 320 may extend longitudinally as shown in FIG. 11A, with the strap 397 attached to balloons 320 on opposing sides of the inflation device 200. The inflation tubes 370 extend from proximal ends of the balloons 320. In other embodiments, the inflation device 400 may have balloons 420 disposed horizontally, as shown in FIG. 11B, with the strap 497 connecting distal ends of the balloons 420 to portions of inflation tubes 470 extending from proximal ends of the balloons 420. In some embodiments, the inflation tubes 470 may extend partially through the strap 497. In either embodiment, the strap 397, 497 may be permanently attached to one side of the inflation device 300, 400 and may be releasably attachable to the opposite side with a snap, hook and loop, tie, or other suitable releasable connector. In other embodiments, the strap 397, 497 may be permanently attached to one side of the inflation device 300, 400 prior to implantation, and permanently attached to the opposite side of the device during implantation, using adhesive, heat welding, or other suitable attachment element. The ability to inflate each is balloon 320, 420 independently allows the choice of certain regions of the heart to be depressed as required by the extent of deficiency in wall motion such as in heart failure. This inflation device 300, 400 may be used acutely to provide emergency circulation, or for post acute myocardial infarction patients to allow the myocardium to rest and repair and recover. In other embodiments, because the device may be implanted through a small incision in the chest, and the catheter may exit through this small incision, the inflation device 300, 400 may be used over a number of weeks as a bridge to transplant or while waiting for a suitable donor. As a permanent implant the inflation tubes 370, 470 may be detached from the balloons 320, 420 with the balloons 320, 420 remaining in their chosen inflated state. This would be beneficial for heart failure patients where a reduction in size of the left ventricle (LV) or an external epicardial wall support enables better LV function.

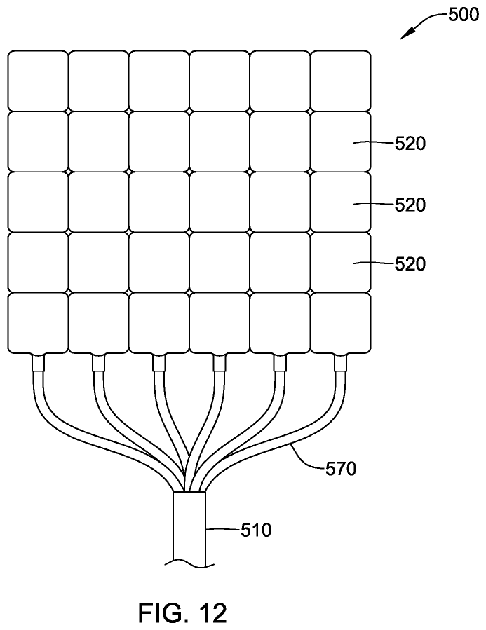

FIG. 12 illustrates another embodiment of inflation device 500 in which the balloons 520 are formed in a grid, for example a 6×5 grid as shown. In some embodiments, the grid may be a rectangle as illustrated and may include any number of balloons, such as 2×3, 3×5, 4×6, etc. In other embodiments the grid may be a square or other geometric shape, having, for example balloons in a grid of 2×2, 3×3, 4×4, 5×5, etc. The above examples are not limiting. Each balloon 520 may be attached to a separate inflation tube 570 that extends into a catheter 510. Only the inflation tubes 570 for the bottom row of balloons 520 are illustrated in FIG. 12 for clarity. The inflation tubes 570 for the remaining balloons 520 may be attached to the back side of the balloons 520. Each of the balloons 520 may be selectively and individually inflated and deflated. In some embodiments, all of the balloons 520 will be inflated simultaneously, while in other embodiments, the balloons 520 may be inflated sequentially in a pattern. In still other embodiments, the balloons 520 may be inflated and deflated to create a wave pattern as discussed above. The grid of balloons 520 may allow for a greater variety in inflation/deflation patterns. The inflation and/or deflation of each balloon 520 may be adjusted only at the time of implantation, or may be adjusted as desired post-implantation through a separate valve disposed within each inflation tube 570. In embodiments in which the inflation device 500 is to be a permanent implant, the valves may be disposed at the proximal ends of each balloon 520 such that the inflation tubes 570 may be disconnected and removed from the body, leaving the inflated balloons 520 in the body.

A method of forming an abdominal pouch 44 within an abdominal wall 20 will be described with reference to the inflation device 200 illustrated in FIG. 4, however it will be understood that the method may be performed in a similar manner with the inflation device 500 illustrated in FIG. 12. The method may include compressing side or laterally outward balloons 244 of the inflation device 200 against the center balloon 242. In some embodiments, an outer sleeve 260, such as an elastic sleeve, may be disposed around the balloons 220. The inflation device 200 may be inserted through the catheter 210 having a hub 205 with a hemostasis valve 214. The catheter 210 is inserted through an incision in the skin of a patient and between a first layer of tissue of the abdominal wall 20 and a second layer of tissue of the abdominal wall 20 immediately adjacent the first layer of tissue (e.g., the parietal peritoneum 40 and the transversalis fascia 42). In some embodiments, as the inflation device 200 is advanced through the hemostasis valve 214, the outer sleeve 260 may be retained within the hub 205. In other embodiments, the outer sleeve 260 is configured to advance through the hemostasis valve 214 around the inflation device 200.

Once the inflation device 200 is in the desired position, the operator may selectively and individually inflate each of the balloons 220 with an inflation media in a desired pattern to achieve the desired pouch 44. For example, the center balloon 242 may be inflated first followed by the laterally outward balloons 244, thereby slowly separating the first layer of tissue from the second layer of tissue to form the abdominal pouch 44 within the abdominal wall 20. In some embodiments not all of the balloons 220 may be inflated, depending on the size and shape of the pouch 44 desired. In at least some embodiments, the inflation media may be cooled to reduce and/or minimize inflammatory reaction(s) by the tissue layers of the parietal peritoneum 40 and the transversalis fascia 42 as the inflatable balloons 220 are expanded to dissect the tissue layers of the parietal peritoneum 40 and the transversalis fascia 42 and form the abdominal pouch 44.

The feature of the balloons 220 being selectively and individually inflatable allows the operator to use a standard inflation device 200 but create a variety of sizes and shapes of pouch 44. In some embodiments. all of the balloons 220 may be inflated simultaneously. If the inflation device 200 is temporary, the balloons 220 are deflated and the device is withdrawn through the catheter 210. After forming the abdominal pouch 44 within the abdominal wall 20, the method may further include deflating the inflatable balloons 220 within the abdominal wall 20, and withdrawing the inflation device 200 from the abdominal wall 20. The abdominal pouch 44 may thereafter be used to accept and/or accommodate a medical implant, such as a diabetes reversing implant, a pacemaker, a defibrillator, or other suitable device. The location of the pouch between the parietal peritoneum 40 and the transversalis fascia 42 may facilitate vascularization of the area following implantation, to facilitate transfer of treatment drugs, particles, and/or other elements from the medical implant into the body and/or bloodstream, for example. If the inflation device 200 is to remain in place in the inflated state, the inflation tubes 270 may be disconnected and removed from the body, leaving the balloons 220 in the body.

Some suitable but non-limiting materials that can be used for the various components of the inflation device 200, the catheter 210, the inflatable balloons 220, the outer sleeve 260, etc. (and/or other systems disclosed herein) and the various elements thereof disclosed herein may include those commonly associated with medical devices.

In some embodiments, the inflation device 200, the catheter 210, the outer sleeve 260, etc., and/or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other is suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the is linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the inflation device 200, the catheter 210, the inflatable balloons 220, the outer sleeve 260, etc., and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the inflation device 200, the catheter 210, the inflatable balloons 220, etc. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the inflation device 200, the catheter 210, the inflatable balloons 220, the outer sleeve 260, etc. to achieve the same result.

In some embodiments, a degree of Magnetic Resonance Imaging (MRI) compatibility is imparted into the inflation device 200, the catheter 210, the inflatable balloons 220, the outer sleeve 260, etc. For example, inflation device 200, the catheter 210, the inflatable balloons 220, the outer sleeve 260, etc., and/or components or portions thereof, may be made of a material that does not substantially distort the image and create substantial artifacts (e.g., gaps in the image). Certain ferromagnetic materials, for example, is may not be suitable because they may create artifacts in an MRI image. The inflation device 200, the catheter 210, the inflatable balloons 220, the outer sleeve 260, etc., or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the inflation device 200, the catheter 210, the inflatable balloons 220, the outer sleeve 260, etc., and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some is embodiments the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. An inflation device, comprising:
a plurality of independently inflatable balloons attached to one another, the balloons configured to move between a deflated, compressed state and an inflated, expanded state; and
a plurality of inflation tubes connected to the plurality of inflatable balloons, wherein each inflation tube is attached to a single one of the plurality of inflatable balloons.

2. The inflation device of claim 1, further comprising a plurality of valves configured to control inflation and deflation of each balloon independently, wherein the valves are disposed either within each balloon or within each inflation tube.

3. The inflation device of claim 1, wherein the valves are disposed within each balloon.

4. The inflation device of claim 1, wherein each balloon defines a single internal inflation chamber.

5. The inflation device of claim 4, wherein the plurality of inflatable balloons includes a center balloon and at least one laterally outward balloon attached to either side of the center balloon, wherein the internal inflation chamber of the center balloon has a generally circular cross-section and the internal inflation chambers of the laterally outward balloons have an elliptical cross-section.

6. The inflation device of claim 5, wherein the elliptical cross-section has a major dimension greater than a diameter of the center balloon internal chamber, and wherein the major dimension increases in each successively further laterally outward balloon.

7. The inflation device of claim 1, wherein the plurality of inflatable balloons each have a distal end, a proximal end, and a side surface, wherein the inflation tubes are attached to the proximal ends and adjacent balloons are attached to one another along their side surfaces.

8. The inflation device of claim 1, wherein the plurality of inflatable balloons are attached to one another in a plane, with each balloon attached to no more than two adjacent balloons.

9. The inflation device of claim 1, wherein the plurality of inflatable balloons are arranged with a center balloon distal end extending further distally than distal ends of adjacent balloons.

10. The inflation device of claim 1, wherein the plurality of inflatable balloons are compliant.

11. The inflation device of claim 1, further comprising at least one guidewire lumen extending through one of the plurality of inflatable balloons or extending through a region of attachment between adjacent balloons.

12. The inflation device of claim 1, further comprising an outer sleeve disposed around the plurality of inflatable balloons.

13. The inflation device of claim 1, further comprising a connector element configured to attach the inflation device to a body lumen or organ, wherein the connector element connects distal ends of the inflatable balloons to proximal ends of the inflatable balloons or the connector element connects a side surface of a leftmost balloon to a side surface of a rightmost balloon.

14. The inflation device of claim 1, wherein the plurality of inflatable balloons defines at least a 2×2 grid of balloons.

15. An inflation device, comprising:
a plurality of laterally adjacent independently inflatable balloons configured to be expanded from a collapsed delivery configuration to an expanded configuration having a generally flattened profile, the inflatable balloons including a series of longitudinally-oriented elongate balloons attached to one another along side surfaces thereof, the balloons including a center balloon and at least two side balloons attached to opposite sides of the center balloon;
a plurality of inflation tubes, each tube connected to one of the plurality of balloons; and
at least one guidewire lumen extending through one of the plurality of inflatable balloons or extending through a region of attachment between adjacent balloons.

16. The inflation device of claim 15, further comprising a plurality of valves configured to control inflation and deflation of each balloon independently, wherein the valves are disposed either within each balloon or within each inflation tube.

17. The inflation device of claim 15, wherein the inflation tubes are attached to proximal ends of each balloon.

18. The inflation device of claim 15, wherein the plurality of inflatable balloons are arranged with a distal end of the center balloon extending further distally than distal ends of the at least two side balloons.

19. The inflation device of claim 15, further comprising an outer sleeve disposed around the plurality of inflatable balloons.

20. A method of forming a pouch within an abdominal wall, comprising:
inserting an inflation device between a first layer of tissue of the abdominal wall and a second layer of tissue of the abdominal wall immediately adjacent the first layer of tissue, the inflation device comprising:
a plurality of independently inflatable balloons attached to one another, the balloons configured to move between a deflated, compressed state and an inflated, expanded state; and
a plurality of inflation tubes connected to the plurality of inflatable balloons, wherein each inflation tube is attached to a single one of the plurality of inflatable balloons;
positioning the inflation device at a desired location of the pouch within the abdominal wall;
selectively and individually inflating the inflatable balloons within the abdominal wall, thereby separating the first layer of tissue from the second layer of tissue to form the pouch within the abdominal wall;
deflating the inflatable balloons within the abdominal wall; and
withdrawing the inflation device from the abdominal wall.
